(19) United States
(12) Patent Application Publication
(10) Pub. No.: US 2003/0039668 A1
(43) Pub. Date: Feb. 27, 2003

(54) Trans dermal skin care

(75) Inventors: Michael Gulla, Sarasota, FL (US); Robert L. Goldberg, Sharon, MA (US)

Correspondence Address: Robert L. Goldberg, 56 Wilshire Street, Sharon, MA, 02067, US

(73) Assignee: Neo Tech Development Company, L.L.C., Sharon, MA

(21) Appl. No.: 10/086,990

(22) Filed: Mar. 1, 2002

Related U.S. Application Data
(60) Provisional application No. 60/274,359, filed on Mar. 8, 2001.

(51) Int. Cl. A61K 7/00

(52) U.S. Cl. 424/401

(57) ABSTRACT

This invention is for a rapid penetrating skin treatment emulsion characterized by an ability to very rapidly penetrate into the epidermis and dermis carrying efficacious additives with it as it penetrates into the skin. The ability to penetrate into the skin is due to the physical structure of the emulsion, which in turn is as a consequence of its ingredients, and the concentration of each ingredient.

### BACKGROUND OF THE INVENTION

1. Introduction

This invention relates to a rapid penetrating skin treatment emulsion suitable for use both as a moisturizer and as a carrier for additional efficacious additives. The emulsion is characterized by a combination of specific components used in specific concentrations that enable the emulsion to penetrate into the epidermis and dermis.

2. Description of the Prior Art

Skin moisturizers represent an important sector of the cosmetic product industry. They are often sold in the form of an emulsion, for example, in the form of a lotion, typically an oil in water emulsion. Many such emulsions contain components known to be beneficial to the skin such as aloe, humectants, medications, etc. Skin moisturizing compositions are typically either topical or penetrating.

Most skin moisturizers sold commercially are topical and fail to substantially absorb into the epidermis and dermis. As a consequence, such moisturizers remain on the surface of the skin and feel greasy, often creating a feeling of discomfort. Moreover, and perhaps more importantly, because the emulsion fails to penetrate into the epidermis and dermis, the emulsion fails to deliver efficacious additives contained within the emulsion into the epidermis and dermis where they would otherwise provide the greatest benefit.

Penetrating or absorbing skin moisturizing formulations are also known. For example, U.S. Pat. Nos. 3,472,931; 3,551,554; and 4,006,218 respectively describe the use of dimethylsulfoxide (DMSO), dimethyl formamide (DMF) and N,N-dimethylacetamide (DMA) to enhance the absorption of topically applied materials through the stratum corneum. Other compounds which have been used to enhance skin permeability include decylmethylsulfoxide, polyethylene glycol monolaurate as disclosed in U.S. Pat. Nos. 4,568,343, and the 1-substituted azacycloheptan-2-ones, particularly 1-n-dodecylcyclazacycloheptan-2-one as disclosed in U.S. Pat. Nos. 3,989,816; 4,316,893; and 4,405,616. Such materials aid penetration into the dermis, but are synthetic materials and as such, have unknown long term effect on the skin and body. In addition, when efficacious additives are included in the formulations, these additives often interfere with absorption of the formulation.

Lecithin is a naturally occurring material contained within many cosmetic products. Lecithin is commercially available as a waxy flake and must be altered for use in a cosmetic. Lecithin is also sold in the form of a solution in an oil base. Typically, the lecithin content varies between about 35 and 65 percent of the total solution. When applied to the skin, the oil-based lecithin forms a greasy, non-penetrating coating over the surface of the skin. However, it has been disclosed in the prior art that lecithin is capable of enhancing penetration of a material into the skin. Consequently, to form an absorbing base formulation, the lecithin must be admixed with other components.

The use of lecithin in a skin treatment composition is disclosed, for example, by Oleniacz in U.S. Pat. No. 3,957,971. Oleniacz discloses moisturizing units for treating keratinous tissue comprising liposomes having a ternary lipid mixture of lecithin, dicetyl phosphate, and a sterol. However, Oleniacz does not discuss absorption of the composition into the skin and it is believed that the formulations of Olenicz do not have the capability to penetrate into the epidermis.

In U.S. Pat. No. 4,481,185 to Grollier et al., it is disclosed that natural emulsifiers such as lecithin or the saponosides, when combined with “aloe juice”, provide emulsions that do not break, even after long periods of storage. Accordingly, the invention disclosed in the patent comprises a cosmetic oil in water type emulsion cream containing a water phase, an oil phase, a natural emulsifier, and a stabilizer, the emulsifier—stabilizer combination comprising 3 to 30 weight percent lecithin or a saponoside, and 0.3 to 30 percent aloe juice. The patent is silent on absorption of the formulation into the epidermis.

In U.S. Pat. No. 4,783,450, Fawzi et al disclose the use of lecithin as a skin penetration enhancer for trans dermal delivery of the drug procaterol through skin. The lecithin is used primarily to enhance the penetration of the drug into the mucous membrane. Cosmetic formulations using lecithin to enhance epidermis penetration are not disclosed in the patent.

Sakai et al., in U.S. Pat. No. 4,760,096, disclose a skin moisturizing method and preparation containing a combination of a phosphatide such as soy lecithin, one or more C10 to C30 carboxylic acid sterol esters and triglycerides. In accordance with the patent, the formulation is useful for treatment of physiological effects, including moisturization, softening and increasing flexibility. Rapid absorption of the composition into the epidermis is not discussed.

In U.S. Pat. No. 4,701,471, Loucks discloses a cosmetic and pharmaceutical composition comprising bovine bone marrow acids mixed with lecithin for prevention of fatty acid oxidation and odor putrefaction. Loucks does not recognize the use of lecithin to assist in trans dermal penetration nor does Loucks suggest any formulation that could be used as a trans dermal moisturizing emulsion.

In U.S. Pat. No. 5,215,759 to Mauner, a cosmetic composition is disclosed comprising water; and emulsified into the water: (1) a moisturizing component comprising: (a) hydrophilic microcapsules and (b) lipophilic microcapsules comprising glycosphingolipids, phospholipids, cholesterol, and at least one long-chain saturated fatty acid; (2) a short-chain fatty acid ester of tocopherol; (3) a glyceryl ester complex; (4) aloe vera gel; and (5) chamomile extract. Each of these ingredients is said to be present in a cosmetically effective quantity. The proportion of hydrophilic microcapsules to lipophilic microcapsules in the moisturizing component is from about 2:3 to about 3:2. The composition preferably further comprises microcapsules comprising methylsilanol elastinate for firming activity, and can additionally comprise caffeine plus microcapsules comprising methylsilanol theophyllinacetate alginate and methylsilanol mannuronate for anti-cellulite activity, along with a number of plant extracts and plant extract-miscible components. The patent appears to be silent on trans dermal penetration of the formulation into the epidermis.

In U.S. Pat. No. 5,738,856, Korb et al disclose a topical treatment composition for skin disorders comprising a compound in a pharmaceutically acceptable carrier for topical delivery that penetrates the epithelial surface into the stratum corneum to provide prolonged lubrication and moistening. The invention is said to be predicated upon the discovery that to provide prolonged lubrication and moistening of the skin, it is desirable to artificially replicate and replenish the bilayer lamellae naturally occurring in healthy skin. In accordance with the stated invention of Korb et al, a topical treatment formulation comprises a pharmaceutically acceptable topical carrier containing a non-toxic compound having one or more polar terminus groups and one or more non-polar terminus groups where the polar and non-polar groups are separated from each other by a spacer segment. The composition is said to comprise a pharmaceutically acceptable carrier, an efficacious amount of a neutral oil, and an efficacious amount of a bilayer component that is a mixture of a negatively charged phospholipid and a triglyceride. The composition is said to be intended to penetrate into the epidermis. In the examples, absorption time is measured in minutes.

Crandall, in U.S. Pat. No. 6,316,428, describes a method for moisturizing keratinous tissue of a human or animal comprising the step of topically applying to the keratinous tissue of the human or animal, a composition comprising lecithin in an organic solvent and water whereby the composition is delivered into the stratum corneum, epidermis and dermis. The composition contains any of an antimicrobial, antibacterial, antifungal, antiprotozoal, or antiviral agent. One aspect of the invention described by Crandall is the addition of poloxamer 407 to the formulation, a polyoxypropylene-polyoxyethylene block copolymer described by Schmolka in the Journal of Biomedical Materials Research 6:571-582, 1972. The patent does not describe the rate of absorption into the skin and it is believed that the presence of organic solvents can cause skin irritation.

In Canadian Patent No. 1,113,427 to Frost et al, a combination of lecithin and an alkanolamine is disclosed and said to be a penetration formulation for treatment of the skin and nails. However, the formulation does not quantitatively define the rate at which absorption occurs.

In Perry, Reverse The Aging Process of Your Face, Avery Publishing Group, New York, 1995, pp. 37 to 51, there is a generalized discussion of formulations for treating the skin and Perry suggests the combination of lecithin and vegetable oils. Though Perry suggests that the formulations absorb, neither quantitative data nor specific formulations are given.

Each of the aforesaid references is incorporated herein by reference for their teachings of lecithin, the use of lecithin in cosmetic formulations, the preparation and formulations of cosmetic products, and the generalized composition of such products.

As described above, it is desirable to include efficacious additives in a skin treatment composition. For example, various efficacious additives include anti-wrinkle, anti-skin atrophy and skin repair actives to replenish or rejuvenate the epidermal layer; skin barrier repair actives which can help repair and replenish the natural moisture barrier function of the epidermis; non-steroidal cosmetic soothing actives; artificial tanning actives and accelerators; sunscreen actives; skin lightening actives; sebum stimulators and inhibitors; protease inhibitors; anti itch additives; hair growth inhibitors; 5-alpha reductase inhibitors; desquamating enzyme enhancers; anti-glycation agents; natural additives such as aloe, etc.

Of those additives identified above, a particularly important component contained in many cosmetic formulations is aloe as there are many ingredients within aloe believed to provide benefits to the skin and to the body as a whole. For example, most aloes comprise amino acids, inclusive of 20 of the 22 human required amino acids and 7 out of 8 of the essential amino acids; anthraquinones, inclusive of aloe emodin, aloetic acid, aloin, anthracine, antranol, barbaloin, chrysophanic acid, emodin, ethereal oil, esters of cinnamonic acid, isobarbaloin, and resistannol, all known for analgesic, anti-bacterial, anti-fungal and anti-viral activity; 8 enzymes, inclusive of aliiase, alkaline phosphatase, amylase, carboxypeptidase, catalase, cellulase, lipase and peroxidase, all known for nutrient absorption properties; the hormones auxins and gibberellins known for wound healing and anti-inflammatory properties; lignin thought to provide penetrating power for skin preparation products and to function as a carrier for other materials; 9 minerals, inclusive of calcium, chromium, copper, iron, magnesium, manganese, potassium, sodium, and zinc, all essential for good health; salicylic acid, an analgesic; glycosides, materials known for cleansing and antiseptic properties; sterols, inclusive of cholesterol, campesterol, lupeol and beta sitosterol, all known as anti-inflammatory agents with beta lupeol also know for antiseptic and analgesic properties; sugars, inclusive of the polysaccharides, glucose and fructose, and the polysaccarides inclusive of gluco-mannans and polymannose, all known for anti-inflammatory and anti-viral activity; the vitamins A, C, E, B, choline, B12, and folic acid, known as antioxidants and of course, a preponderance of water required for moisturizing dry skin. In addition to the above advantages obtained from the use of aloe, components within aloe have an ultraviolet absorption spectrum which exhibit a peak at about 290 nm-300 nm, the erythematous region. Therefore, aloe has anti-sunburn properties that have been described by G. Proserpio, Cosmetics and Toiletries, Volume 91, March 1976. Consequently, aloe-based emulsions are suitable for use as anti-sunburn products.

Cosmetic formulations containing lecithin and efficacious additives are known from the above cited prior art. Many of the formulations shown in the above cited prior art are topical without the ability to penetrate into the epidermis or dermis. Other formulations shown in the prior art do have the capability of penetrating into the epidermis and dermis notwithstanding that lecithin itself is incapable of such penetration. However, it is believed that the addition of many desired efficacious additives to a penetrating lecithin based cosmetic delivery system, particularly in significant concentration, interferes with or prevents the rapid penetration of the cosmetic into the epidermis thus converting a penetrating base to a topical non-penetrating formulation, often having a greasy and unpleasant feel. Accordingly, it would be desirable to provide a lecithin based, rapid penetrating cosmetic formulation to which a wide variety of efficacious additive components may be incorporated into the composition in significant concentration without diminution of the penetrating capability of the base formulation.

### SUMMARY OF THE INVENTION

In accordance with the subject invention, it has been unexpectedly discovered that the combination of lecithin, or the phosphatides within lecithin, or a saponoside; combined with one or more surfactants, preferably one or more essential oils, one or more gel formers, and water, all in controlled concentration, can be combined to form a high water content emulsion that is capable of rapid penetration into the epidermis, and further capable of carrying with it a high concentration of water soluble efficacious additives. Typically, the emulsions of the invention are capable of essentially complete absorption into the epidermis such that the skin is dry to the touch within a period of less than sixty seconds, and often, within a period of less than thirty seconds, even when the concentration of water and the efficacious additives within the emulsion are as high as eighty percent by weight of the total emulsion.

For purposes of the discussion that follows, in one embodiment of the invention, the emulsion of the invention will be considered a base carrier formulation and will comprise two primary categories of components. In a second embodiment of the invention, the emulsion will be considered a base carrier containing efficacious additives and will be considered to contain three primary categories of components.

The composition of the first embodiment of the invention comprises:

1. oil phase constituents in a concentration of from 3 to 35 weight percent of the total emulsion, the oil base constituents comprising:

a. lecithin, phosphatides, or saponosides in a concentration of from 1 to 10 percent by weight of the emulsion and preferably in a concentration of from 1 to 6 percent of the emulsion;

b. one or more essential oils in a concentration sufficient to form the emulsion, typically from 2 to 15 percent by weight of the emulsion and preferably in a concentration of from 3 to 8 percent of the emulsion;

c. one or more gel formers in a concentration of from 0.1 to 5 percent by weight of the emulsion and preferably in a concentration of from 1 to 3 percent of the emulsion; and

d. one or more surfactants in a concentration of from 0.1 to 5 percent by weight of the emulsion and preferably in a concentration of from 0.5 to 3 percent of the emulsion;

2. water as an aqueous phase comprising the balance of the formulation.

The composition that defines the second embodiment of the invention comprises:

1. oil phase constituents in a concentration of from 3 to 20 weight percent of the total emulsion, the oil base constituents comprising:

a. lecithin, phosphatides, or saponosides in a concentration of from 2 to 10 percent by weight of the emulsion and preferably in a concentration of from 3 to 7 percent of the emulsion;

b. one or more essential oils in a concentration sufficient to form a stable emulsion, typically from 1 to 10 percent by weight of the emulsion and preferably in a concentration of from 3 to 7 percent of the emulsion;

c. one or more gel formers in a concentration of from 0.1 to 5 percent by weight of the emulsion and preferably in a concentration of from 1 to 3 percent of the emulsion; and

d. one or more surfactants in a concentration of from 0.1 to 5 percent by weight of the emulsion and preferably in a concentration of from 1 to 3 percent of the emulsion;

2. water as the aqueous phase in a concentration of from 40 to 80 weight percent of the emulsion, preferably exceeds 50 per cent of the emulsion and desirably varies between 60 and 75 weight percent of the emulsion.; and

3. efficacious additives comprising the balance of the emulsion where the efficacious additives differ from the oil phase constituents.

The pH of the compositions of both embodiments of the invention preferably varies between 6 and 8 and more preferably, between 6.4 an d 7.6.

The emulsions of the invention are non greasy and capable of rapid penetration into the epidermis and dermis with the further capability of carrying large concentrations of efficacious additives into the epidermis and dermis within periods of time of less than one minute. Consequently, the emulsions of the invention are suitable for use as a cosmetic base composition and as a drug delivery composition.

Without wishing to be bound by theory, and as discussed in greater detail below, it is believed unexpected that an emulsion having the components in the concentrations given form a stable micro emulsion that is drawn into the epidermis even though the individual components of the emulsion are for the most part incapable of absorption into the epidermis.

### DESCRIPTION OF THE PREFERRED EMBODIMENTS

The following discussion describes the preferred embodiment of the invention, an emulsion containing efficacious additives, in detail. However, as above, the invention also contemplates a base carrier composition free of efficacious additives.

The preferred compositions of the invention comprise oil phase constituents, water and efficacious additives. The oil phase constituents comprise lecithin or phosphatides whether obtained from lecithin or synthesized, a saponoside, or a mixture of the two.

Lecithin can be obtained from a variety of sources such as egg yolks, soybeans, etc. as is well known in the art. These sources typically contain a mixture of components including natural lipids as exemplified by glycerides; cholesterol and cholesterol esters; phospholipids or phosphatides having a net charge of zero as exemplified by phosphatidylcholine and phosphatidyl-ethanolamine; various unsaturated and saturated fatty acids; and charged phospholipids such as phosphatidylglycerol and phosphatidylinositol. It is believed that the phospholipids, especially the negatively charged polyol phospholipids, are especially useful for rapid absorption of the formulations of the invention into the skin.

The phospholipids, or phosphatides, are typically contained in naturally occurring lecithin products in amounts of from 30 to 65 percent by weight. The positively charged phospholipids are present in minor concentration, typically varying from below one percent up to 10 to 15 percent of the total lecithin composition. Lecithin products containing a total phosphatide content of at least 35 percent by weight, preferably at least 45 percent by weight, and most preferably, up to 65 percent by weight or more, are preferred for purposes of this invention. Phospholipids obtained from lecithin, or synthetic phospholipids, can be used in place of lecithin, but the cost of the lecithin is substantially less than the cost of the pure phospholipids, and therefore, it is more economical to use lecithin rather than the pure phospholipid. In addition, it is believed that other components contained within commercial liquid lecithin products, such as the neutral lipids, provide benefits to the composition.

The saponosides used with lecithin or as an alternative to lecithin occur naturally within various vegetable substances. The saponosides essentially consist of a saponogen and an ose. The saponogens are generally either of a steroid type or of a triterpene type. Amongst the plants which give extracts in which the saponogens have a steroid structure, there may be mentioned in particular those belonging to the (1) Liliaceae family and especially (a) the Smilax (salsaparilla) species, for example: Smilax aspera L, Smilax officinalis, Smilax regilii, Smilax glaberrina, Smilax medica, aristolochiaefolia, Smilax papyraceae, Smilax febrifuga, Smilax omata, Smilax saluberina and Smilax china; (b) the Asparagus species, for example, Asparagus officinalis L, Asparagus persicus and Asparagus tenufolius; (c) the yuccas, for example, Yucca filifera, Yucca treculeana, Yucca glauca, Yucca filamentosa, Yucca gloriosa and Yucca shottii, and (d) the Ruscus species, for example, Ruscus aculeatus L (butcher's broom); (2) those belonging to the Discoreaceae family especially the Discorea species, for example: Discorea tokoro, Discorea mexicana, Discorea toxicaria and Discorea sylvatica; and (3) Those belonging to the Amaryllidaceae family, especially the Agave species, for example: Agave sisalana and Agave fourcroydes.

Lecithin is preferred to the saponogens for purposes of the subject invention.

Oil is used in the emulsion to stabilize the same, possibly by reinforcement of the walls of the micelles of the emulsion. A preferred oil is a polar oil. The oils should not be used in excess as an excess may interfere with absorption of the emulsion into the skin and create a greasy feel when the emulsion is applied. The most preferred concentration of the oil is the lowest amount needed to form an absorbing emulsion.

Though non-polar oils such as mineral oil may be used, vegetable oils and hydrogenated vegetable oils are preferred. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, flaxseed oil, linseed oil, rice bran oil, pine oil, sesame oil, sunflower seed oil, hydrogenated safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated soybean oil, hydrogenated rapeseed oil, hydrogenated linseed oil, hydrogenated rice bran oil, hydrogenated sesame oil, hydrogenated sunflower seed oil, almond oil, avocado oil, and mixtures thereof. Synthetic oils are also suitable such as ethyl palmitate, isopropyl palmitate, alkyl myristates, such as isopropyl myristate, butyl myristate and decyl myristate, hexyl stearate, triglycerides such as triglycerides of tanoic and decanoic acid, cetyl ricinoleate, stearyl octanoate (Purcellin oil) and hydrogenated polyisobutene. Vegetable oils are preferred with soybean oil and flaxseed oil are the most preferred oils. It should be noted that many sources of soy lecithin are sold in the form of an oil solution of lecithin in soybean oil and such combinations are most preferred for purposes of this invention.

Another component of the oil phase constituents is one or more gel formers. As will be explained in greater detail below, the concentration of the gel former is desirably a concentration that provides a rigid, closely packed micelle. Gelling agents vary greatly in there ability to thicken an emulsion and therefore the concentration range given above should be interpreted as a guideline only with the selected amount being that amount which thickens the emulsion as described.

Suitable gel formers include for example, clays, polysaccharide gums and their derivatives such as xanthan gum, carboxymethylhydroxypropyl guar, or carboxyvinyl polymers, fatty acid metal salts such as aluminum stearates, hydrophobic silica, polyethylenes, and ethylcellulose, and synthetic cationic polymers include, for example: copolymers of 1-vinyl-2-pyrrolidone and 1-vinyl-3-methylimidazolium salt (e.g., chloride salt) such as those commercially available from BASF Wyandotte Corp. (Parsippany, N.J., USA) under the LUVIQUAT tradename, copolymers of 1-vinyl-2-pyrrohdone and dimethylaminoethyl methacrylate such as those described in U.S. Pat. No. 4,080,310 and commercially available from ISP Corporation (Wayne, N.J., USA) under the GAFQUAT tradename; cationic diallyl quaternary ammonium-containing polymers, including, for example, dimethyldiallylammonium chloride homopolymer and copolymers of acrylamide and dimethyldiallylammonium chloride; the mineral acid salts of amino-alkyl esters of homo- and co-polymers of unsaturated carboxylic acids having from 3 to 5 carbon atoms, as described in U.S. Pat. No. 4,009,256; the graft cationic copolymer containing N-vinylpyrrolidone, dimethylaminoethyl methacrylate and polyethylene glycol described in U.S. Pat. No. 4,048,301; the polymers of etherified starch described in U.S. Pat. No. 3,186,911; cationic polyacrylamides of the type described in British Patent App. 94031565.4; the high molecular weight cationic polymers Quaternium-40 polymers such as dimethyldiallylammonium chloride homopolymer) and Quaternium-41 such as dimethyldiallylammonium chloride and acrylamide), which are commercially available under the trademarks MERQUAT 100 and MERQUAT 550 from Merck & Corn., Inc.; and mixtures thereof. Further examples of other suitable synthetic polymers include acrylates/C10-30 alkyl acrylate crosspolymer, acrylates copolymer, acrylates/PVP copolymer, acrylates/VA copolymer, butylated polyoxymethylene urea, butylated PVP, carbomer, hydroxyethyl PEI-1000, methyl methacrylate crosspolymer, PEI-1000, PEI-1500, PEI-2500, polybutene, polyacrylamide, polyacrylic acid, polyethylene, polyisobutene, polymethyl methacrylate, polystyrene, polyvinyl alcohol, PVP, PVP/Eicosene copolymer, PVP/VA copolymer, sodium acrylates copolymer, sodium carbomer, sodium polyacrylate, sodium polymethacrylate, styrene/PVP copolymer, TEA carbomer, high molecular weight acrylic acid polymers having polyalkenyl polyether crosslinking agents sold under the tradename Carbopol such as Carbopol 940, Carbopol 941, Carbopol 934, Carbopol Ultrez 10 and Carbopol ETD020202, all available from B. F. Goodrich, and mixtures thereof.

Of the above, the Carbomers and Carbopols are preferred with Carbopol Ultrez 10 most preferred for its ability to greatly increase the viscosity of a liquid in which it is dissolved even when present in low concentration. As briefly indicated above, and as will be explained in greater detail below, a low concentration of the gel former is desired to form a tightly packed micro gel capable of penetrating into the epidermis and dermis.

Lecithin is a known emulsifier. However, it has been found desirable to add an additional emulsifier. Therefore, the oil phase constituents desirably include a surfactant in addition to lecithin. The surfactant used in the composition of the invention assists in the formation and stabilization of the emulsion. The surfactant may be selected from a wide variety of ionic, amphoteric and non-ionic materials. Examples of suitable materials include polyoxyethylenated derivatives of cetyl, stearyl and/or lauryl alcohol, esters of sorbitan and of stearic acid, esters of sorbitan and of palmitic acid, esters of sucrose and of stearic acid, esters of polyethylene glycol and of stearic acid, esters of glycerol or of polyglycerol and of stearic acid, and mixtures thereof.

Suitable commercial surfactants include the following compounds (under the CTFA name, International Cosmetic Ingredient Dictionary and Handbook): ceteth-2 (for example Brij 52 sold by ICI), steareth-2 (for example Brij 72 sold by ICI), sorbitan palmitate (for example Span 40 sold by ICI), sorbitan stearate (for example Span 60 sold by ICI), sorbitan tristearate (for example Span 65 sold by ICI), PEG-8 stearate (for example Myij 45 sold by ICI), sucrose distearate which is a mixture of sucrose mono-, di- and triester (for example Crodesta F10, Crodesta F20 and Crodesta F50 sold by Croda), polyglyceryl-2 stearate (for example 10 Nikkol DGMS sold by Nikko), polyglyceryl-2 distearate (for example Emalex PSGA sold by Nikko) and polyglyceryl-3 distearate (for example Emalex DGS 3 sold by Nikko). The preferred surfactants are non-ionic surfactants such as polyoxyethylene sorbitan monolaureate sold under the tradename Tween, especially Tween 20.

The emulsions of the invention are characterized by a relatively high water content. It is believed that a large volume water phase is desirable to dissolve and hold desired efficacious water-soluble additives in the emulsion and enable these components to transverse the skin and move into the epidermis and dermis when the emulsion is applied to the skin. In addition, the more water carried into the epidermis and dermis, the more effective the emulsion will be in its ability to moisturize the skin.

The term “efficacious additives” as used herein means desired additives contained within the emulsion, other than water and the oil phase constituents, that serve specific treatment purposes. The additives comprise the balance of the formulation. It is believed that the emulsions of the invention are capable of carrying larger concentrations of water-soluble efficacious additives than oil soluble additives because of the large volume of water comprising the emulsion. However, lesser concentrations of oil soluble additives may be dissolved in the oil phase of the emulsion or may be contained as further dispersed particles within the emulsion and therefore are suitable additives for purposes of the invention.

The term efficacious additives includes within its scope agents and ingredients commonly used in dermatological and cosmetic ointments and lotions. Suitable additives include, for example, pH adjustors and buffers such as sodium hydroxide, sodium citrate or tetrasodium EDTA; excipients; fragrances such as menthol; opacifiers such as zinc oxide, magnesium aluminum silicate and titanium dioxide; preservatives such as dichlorobenzyl alcohol, benzoic acid, methylparaben and phenyl carbinol; antioxidants; stabilizers; emollients; coloring agents and the like may be present in the emulsion. All of such materials are well known in the art and used for their art recognized purposes.

The most preferred efficacious additive for purposes of the invention is aloe. The advantages of aloe for treatment of the skin have been discussed above. Aloe is often sold in the form of an aloe vera gel for use as a skin treatment cosmetic. When applied to the skin, the gel forms a topical, somewhat greasy feeling film over the surface of the skin. This topical film exhibits limited ability to absorb into the epidermis and remains on the surface as a topical layer for an extended period of time, typically for in excess of five to ten minutes. Many consumers find the feel of the film to be objectionable and often wash the film residue from the skin surface. For the aloe within the gel to provide maximum benefit to the epidermis, it desirably should penetrate into the epidermis where a lack of water within the stratum corneum is the most common cause of dry skin and where the aloe would provide greatest benefit.

The cosmetic industry, taking advantage of the consumer recognized or perceived benefits of aloe, frequently adds aloe as one of many ingredients to cosmetics and advertises the presence of aloe on the product label and in promotional materials for the product. Because the aloe is contained as one component within a complex formulation, it is present in the formulation in minor concentration. Moreover, since aloe is topical and does not penetrate into the skin, the addition of aloe as an additive to a penetrating formulation in any significant concentration results in the aloe interfering with the penetration of the entire formulation into the skin and the benefits arising from a penetrating composition are lost.

It is an unexpected advantage of the invention that aloe may be incorporated into the emulsion of the invention in relatively large concentration without interfering with the ability of the emulsion to rapidly penetrate into the skin, especially where aloe and aloe vera gel, and the remaining components of the emulsions of the invention, are incapable of individual penetration into the epidermis. In accordance with the invention, emulsions containing high concentrations of aloe are capable of absorbing into the skin within a period of less than 1 minute.

In addition to aloe, the emulsions of the invention carry other efficacious additives into the epidermis. Consequently, the emulsions of the invention are ideally suited for use as a cosmetic base for delivery of specific cosmetic and drug delivery additives. As such, they would further include an active agent or drug known to the art to mean any chemical material or compound suitable for topical or topical administration that induces any desired local or systemic affect. Such substances include the broad classes of compounds normally delivered through body surfaces and membranes, including skin, preferably water soluble additives when needed in large concentration. Various classes of such materials and specific examples of members from each class follow.

Anti-wrinkle, anti-skin atrophy and skin repair actives effective in replenishing or rejuvenating the epidermal layer are suitable additives to the emulsions of the invention. These actives generally provide these desirable skin care benefits by promoting or maintaining the natural process of desquamation. Examples include retinoic acid and its derivatives; retinal; retinol; retinyl esters; vitamin B compounds; salicylic acid; sulfur-containing D and L amino acids; thiols; lysophosphatidic acid; skin peel agents such as phenol; adapalene; ademethionine; adenosine; arginine amino benzoate; ascorbic acid; ascorbyl palmitate; asiatic acid; asiaticosides; betulinic acid; biotin; catecholamines; chalcones; citric acid esters; coumestrol; dehydrocholesterol; dehydrodicreosol; dehydrodieugenol; dehydroascorbic acid; dianethole; diglycol guanidine succinate; disodium ascorbyl phosphate; estrogen and its derivatives; ergosterol; eythrobic acid; gamma amino butyric acid; gingko bilboa extracts; ginsenoside; glutathione and its esters; glycitein; hesperitin; hydroxyethyl isostearyloxy isopropanolamine; hypotaurine; lectins; lupenes; luteolin; lysophosphitidic acid; melatonin; metalloproteinase inhibitors; methoprene; N methyl serine; N methyl taurine; pantethine; phenylalanine; photoanethone; piperdine; pratensein; pregnenolone; premarin; quillaic acid; retinyl glucuronate; retinyl linoleate; S-carboxymethyl cysteine; sodium ascorbyl phosphate; tachysterol; taurine; thymus extracts; thyroid hormones; tigogenin; tocopheryl retinoate; toxifolin; traumatic acid; tricholine citrate; uracil derivatives; and mixtures thereof.

An additional class of additives includes skin barrier repair materials which can help repair and replenish the natural moisture barrier function of the epidermis. Examples of skin barrier repair actives include ascorbic acid; biotin; caffeine; campesterol; canola derived sterols; cholesterol; cholesterol hydroxystearate; glyceryl serine amide; hydroxyethyl isostearyl isopropanolamine; lactic acid; lanolin; lanolin alcohols; lanosterol; lipoic acid; N-acetyl cysteine; N-acetyl-L-serine; vitamin B3 compounds; palmitic acid; panthenol; panthetine; phosphodiesterase inhibitors; sitosterol; soybean derived sterols; sphingosine; stearic acid; thioctic acid; trimethyl glycine; tocopheryl nicotinate; vitamin D; and mixtures thereof.

Cosmetic soothing actives can be added to the emulsion and are effective in preventing or treating inflammation of the skin. The soothing active enhances the skin appearance by contributing to a more uniform and acceptable skin tone or color. Examples of cosmetic soothing agents include propionic acid derivatives; biphenylcarboxylic acid derivatives; acetyl salicylic acid; benoxaprofen; flurbiprofen; indoprofen; carprofen; pranoprofen; tioxaprofen; bucloxic acid; acacia; borage oil; bromelain; calendula; capsicum; chamomile; chirata; comfrey; epimedium; ethacrynic acid; ganoderma; gaoben; gentian; ginseng extract; hypericum; ichthyol; ipecac; melatonin; mono or diglucosides of glabridin; papain; stearyl glycyrrhetinate; tocopheryl acetate; witch hazel; yeast extract; yucca, and mixtures thereof.

An additional class of actives comprises artificial tanning materials and accelerators to help in simulating a natural suntan by increasing melanin in the skin or by producing the appearance of increased melanin in the skin. Examples of artificial tanning agents and accelerators include tyrosine; acetyl tyrosine; coffee extracts; dihydroxyacetone; isobutyl methyl xanthine; methyl xanthine; prostaglandins; theophylline; tyrosine;

Skin lightening actives comprise an additional class of additives for the formulations of the invention. Skin lightening actives can actually decrease the amount of melanin in the skin or provide such an effect by another mechanism. Examples of skin lightening actives include adapalene; alpha-glycaryl-L-ascorbic acid; ammonium lactate; anethole derivatives; arbutin; ascorbic acid; ascorbyl palmitate; butyl hydroxy anisole; 1,3 diphenyl propane derivatives; 2,5 dihydroxybenzoic acid; escinol; esculoside; esculetin; gallic acid and its derivatives; genistic acid and its derivatives; gluco pyranosyl-1-ascorbate; gluconic acid; glucosamine; glycolic acid; hydroquinine; 4 hydroxyanisole; 4-hydroxy benzoic acid; inositol ascorbate; linoleic acid; 5-octanoyl salicylic acid; pyrogallol derivatives; retinoic acid; retinol; salicylic acid; 3,4,5 trihydroxybenzyl; vitamin D and its analogs, and mixtures thereof.

Also useful herein are sun-screening actives. Examples of sunscreens which are useful in the emulsions of the present invention are those selected from the group consisting of aminobenzoic acid; 2-ethylhexyl p-methoxycinnamate; 2-ethylhexyl N,N-dimethyl-p-aminobenzoate; p-aminobenzoic acid; butyl methoxy dibenzoyl methane; diethanolamine p-methoxycinnamate; dioxybenzone; glyceryl aminobenzoate; homomenthyl salicylate; 2-phenylbenzimidazole-5-sulfonic acid; oxybenzone; octyl salicylate; 3-benzylidene camphor; and mixtures thereof.

Sebum stimulators can increase the production of sebum by the sebaceous glands. These skin care actives are especially useful for postmenopausal women who are sebum deficient. Examples of sebum stimulating actives include bryonolic acid, dehydroetiandrosterone, orizanol and mixtures thereof. Sebum inhibitors can decrease the production of sebum by the sebaceous glands. Examples of sebum inhibiting actives include aluminum hydroxy chloride, corticosteroids, dehydroacetic acid and its salts, dichlorophenyl imidazoldioxolan, gugulipiu, ketoconazole, niacinamide, phloretin, sepicontrol AS, spironolactone, tioxolone, tocopherol, tranexamic acid, and mixtures thereof.

An additional class of actives comprises protease inhibitors. Examples of protease inhibitors which are useful are those selected from the group consisting of allicin; alpha lupaline; areca catechu extract; cholesterol sulfate; uncaria gambis roxburgh extract; and mixtures thereof.

Skin tightening agents are a particularly valuable class of additives for the emulsions of the invention. Examples of skin tightening agents comprise egg albumen; Flexan 130 (available from National Starch); Gatuline Lifting (available from Gattefosse); Pentacare HP (available from Pentapharm); Vegeseryl (available from Laboratories Serobioloques) and mixtures thereof.

Hair growth inhibitors and hair growth stimulators can be used as components of the emulsions of the invention. Hair growth inhibitors and include 17-beta estradiol; adamantyguanidines; adamantylamidines; adenylosuccinate synthase inhibitors; anti angiogenic steroids; dehydroepiandrosterone; gamma glutamyl transpeptidase inhibitors; glucose metabolism inhibitors; glutamine metabolism inhibitors; L-aspargine synthase inhibitors; lipoxygenase inhibitors; panthenol; and mixtures thereof. Hair growth stimulators include minoxidril.

Therapeutic agents may also be added to the emulsions of the invention. In general, this includes therapeutic agents in all of the major therapeutic areas including, but not limited to, anti-infectives such as antibiotics and antiviral agents; analgesics and analgesic combinations; anorexics; anti arthritics; anti asthmatic agents; anticonvulsants; antidepressants; antidiabetic agents; antidiarrheals; antihistamines; antiseptics; antiinflammatory agents; antimigraine preparations; antimotion sickness agents; antinauseants; antineoplastic agents; antiparkinsonism drugs; antipruritics; antipsychotics; antipyretics; antispasmodics, including gastrointestinal and preparations, including calcium channel blockers, beta-blockers; antiarrhythmics; antihypertensives; diuretics; vasodilators, including general coronary, peripheral and cerebral; central nervous system stimulants; cough and cold preparations; decongestants; diagnostics; hormones; hypnotics; immunosuppressives; muscle relaxants; parasympatholytics; psychostimulants; sedatives, tranquilizers, anesthetics, vitamins and combinations of the above. The amount of the pharmacologically active agent is an effective amount defined as a non-toxic but sufficient amount of a compound to provide the desired local or systemic effect and performance at a reasonable benefit/risk ratio attending any medical treatment.

The emulsions of the invention are made in a conventional manner. Example 10 below provides the preferred method for making the compositions of the invention.

The compositions are used to treat the skin in essentially the same manner as a conventional skin cream. For use as a moisturizer, the composition is rubbed onto the skin at least once daily and more preferably, twice daily in the morning and evening. More severe skin disorders may require more frequent application. The composition is typically applied to the skin in an amount sufficient to cover the affected area and form a thin coating of the emulsion over the skin. Excessive amounts are not necessary and are undesirable. The composition rapidly passes into the skin, typically within a period of from fifteen seconds to one minute, dependent upon the specific formulation used. Some formulations may take several minutes to penetrate, but such compositions are characterized by high concentrations of efficacious additive.

The compositions of the present invention are also suitable for use as cosmetic compositions, for example, anti-sunburn creams or milks; creams or milks for care or cleaning of the face, body or hands; or moisturizing make-up, foundations or eye make-up; etc.

It is believed that the components of the emulsion in the concentrations given is significant to the ability of the emulsions of the invention to rapidly absorb into the epidermis and dermis carrying with it large concentrations of efficacious additives as it absorbs. The following theory of operation is proffered, but should not be construed as binding.

As shown by the literature, lecithin is known to absorb into the skin and is further known to enhance absorption of extraneous materials into the skin. Moreover, lecithin appears to be capable of bonding with both oil and water. However, as demonstrated by examples 1 and 2 below, solutions of liquid lecithin are incapable of penetrating into the skin, and many formulations containing lecithin, including cosmetics in the form of emulsions and micro emulsions, are incapable of penetrating into the skin notwithstanding the known absorption properties of lecithin. Consequently, it follows that lecithin is capable of absorbing into the skin only if certain conditions are met.

One condition necessary for epidermal penetration of a lecithin-based composition is believed to be the physical form of the lecithin within the composition when applied to the skin. In this respect, it is postulated that the lecithin must be in the form of minute particles or spheres of skin pore or smaller dimension, such as an aerosol or liquid micro emulsion. When lecithin is in the form of an aerosol, it is believed that it readily penetrates into the skin, especially into mucous membrane. However, when lecithin is a component of a cosmetic emulsion, even a micro emulsion, it fails to rapidly penetrate into the skin. Therefore, to obtain an emulsion capable of rapid penetration of the skin, it is believed that there are further physical requirements of the emulsion if the lecithin is to penetrate the skin. In this respect, it is believed that the particle or micelle containing lecithin must be of a given structure and other components of the emulsion must not interfere with or block the lecithin containing micelles from penetrating the skin surface.

Most commercially available cosmetic emulsions containing significant quantities of water are believed to be oil in water emulsions. Since lecithin is an oil soluble material, it is likely to be dissolved in the oil phase of the emulsion. Where the oil phase is the dispersed phase and water is the continuous phase, the emulsion micelles containing lecithin are surrounded by large volumes of the aqueous phase. Notwithstanding the shape, size, or form of the dispersed micelle, when the cosmetic is applied to the skin, the oil phase is separated from the skin surface by a large aqueous interfacial layer. It is believed that this aqueous layer may function as an interfacial barrier layer preventing contact of a significant portion of the lecithin containing micelles with the skin thereby inhibiting rapid penetration of the lecithin into the skin.

If the a major portion of the volume of the aqueous phase is eliminated, then that volume is no longer available as an interfacial barrier to penetration of the oil phase. For example, if the emulsion is a dual emulsion, that is an emulsion having a micelle comprising water contained within an oil film with the micelle dispersed in an aqueous continuous phase, then much of the water is contained within the micelle and eliminated from the continuous phase where it may function as an interfacial barrier. In accordance with the invention, it is believed that either a water in oil emulsion or a dual emulsion is formed dependent upon the water content of the emulsion, the lower water content favoring the water in oil emulsion and the higher water content favoring the dual emulsion.

Regardless of whether the emulsion of the invention is a water in oil emulsion or a dual emulsion, it is believed that there are still additional requirements for rapid absorption of the emulsion into the skin. It is believed that the micelles must be of very small sub pore diameter and must have a structure where lecithin has significant contact with the skin. To have such contact, it is believed that the lecithin should present a large surface area and should not be surrounded or encapsulated by an excessive continuous phase that functions as a barrier preventing substantial contact of lecithin micelles with the skin surface. To prevent encapsulation, it is believed necessary that the emulsion be tightly packed with the walls of the micelles being in close contact with each other.

Given the above discussion, it is believed by selection of the components of the emulsion of the invention in the concentrations given, an emulsion is formed that is either a water in oil emulsion, or a dual emulsion comprising a water droplet having an oil phase film in a continuous water phase. The form of the emulsion is believed to be dependent upon the concentration of the water. For either emulsion, it is believed that it comprises tightly packed micelles having walls in contact with each other, where the micelles are micro spheres of water and dissolved efficacious additives coated with a lecithin membrane, or a lecithin membrane fortified by bonding to the oil in the formulation, further rigidified by the gel former. The gel former is believed to fill a portion of the interstices between the aqueous micelles with micelles of the gel. Moreover, it is known that lecithin is highly hygroscopic. Therefore, it is further believed that the lecithin coating over the aqueous droplet becomes hydrated further bonding the coating to the water droplet.

Using the above model for the emulsion of the invention, oil and lecithin layers coat the micro spheres and therefore is hydrated and has a large surface area. Because the micelles are tightly packed, the micelles having the oil and lecithin coating are in direct contact with the surface of the skin where the lecithin, in contact with the skin, penetrates into the epidermis and dermis together with the aqueous contents of the micro sphere.

The above explanation is consistent with the perceived theory of operation of the emulsions of the invention. Lecithin comprises phosphatides, especially negatively charged polyol phospholipids, as does the membrane surrounding epithelial body cells. It is believed that as the micelles within the emulsion penetrate into the epidermis and dermis, and make contact with cellular material, they bond and reinforce the cell membranes. Since the micelles surround a sphere of water and the lecithin coating is highly hydrated, the water binds to the cell walls and moisturizes the skin. Where efficacious additives are dissolved in the aqueous phase as well as in the oil phase, dependent upon the additive in the emulsion, the efficacious additives are brought to the epithelial cell wall where they provide the greatest benefit.

The following Examples better illustrate the preferred embodiments of the invention. Example 9 represents the best mode of the invention.

### EXAMPLE 1

A thin film of liquid lecithin sold under the trade name Fearn liquid lecithin and identified as a pure vegetable product in its natural liquid unbleached state containing 35% oils was spread on the back of the hand and the time to absorb into the skin was measured. For purposes of the test, absorption is considered complete when the skin to which the formulation is applied is dry to the touch. There was no noticeable absorption of lecithin into the skin within 5 minutes of coating the lecithin onto the skin at which time the test was discontinued.

### EXAMPLE 2

The test of Example 1 was repeated substituting a liquid lecithin obtained from Riceland Products and identified as Leciprime S for the Fearn lecithin used in example 1. Leciprime S contains about 50 to 60 percent lecithin oil. There was no absorption detected after 5 minutes and the test was discontinued.

### EXAMPLE 3

A mixture of 10.8 grams of Leciprime S, 73.3 grams of water, and 2.6 grams of Tween 20 surfactant was prepared. Tween 20 is a polyoxyethylene sorbitan monooleate material available from Wako Chemical Richmond, Va. The formulation was spread on the back of the hand. No absorption was detected within 5 minutes and the test was discontinued.

### EXAMPLE 4

Example 3 was repeated but 1.2 grams of Carbopol Ultrez 10 polymer from B. F. Goodrich was dissolved in water and then 1,2 grams of triethanolamine were added. Carbopol Ultrez 10 is a high molecular weight acrylic acid polymer having polyalkenyl polyether crosslinks. Thereafter, the Tween 20 surfactant was added followed by the Leciprime S. The formulation was spread on the back of the hand and the mixture completely absorbed into the hand in less than 1 minute.

### EXAMPLE 5

A first solution was prepared comprising 1 gram of Carbopol Ultrez 10 polymer, one gram of triethanolamine, and 75 ml of water. A second solution was prepared by mixing 1 gram of dry lecithin flakes with 2 grams of soybean oil. The second solution was mixed into the first solution. The formulation was spread on the back of the hand and the mixture completely absorbed into the hand in less than 1 minute.

### EXAMPLE 6

The test of example 1 was repeated substituting an aloe vera gel for the lecithin. The aloe vera gel used was sold under the trade name Hawaiian Tropic and contained aloe babadensis and minor amounts of conventional additional components such as propylene glycol, glycerin, polysorbate 20, tocopheryl acetate, retinyl palmatate, pantherol, matricaria extract, jojoba oil, carbomer 940, trietheranolamine, diazoolidinyl urea, propylparaban, methylparaban, disodium EDTA, and various fragrances. There was no noticeable absorption of the lecithin into the skin within 5 minutes at which time the test was discontinued.

### EXAMPLE 7

A composition was prepared by gently mixing together 2.5 grams of the lecithin of Example 1 with 25 grams of the aloe vera gel of Example 5. A yellow, smooth viscous emulsion was obtained. The emulsion was rapidly formed and stirring was continued for about 1 minute. The above formulation, used sparingly, was rubbed onto the back of a hand, and again, the time to fully absorb into the skin leaving a dry feel to the skin was measured. The time for full absorption was about 20 seconds. Following application and absorption, the skin had a smooth silky feel.

### EXAMPLE 8

The procedure of Example 7 was repeated with 1 gram of Vitamin E oil added to the formulation. The results were essentially the same as the results for Example 3.

### EXAMPLE 9

The procedure of Example 7 was repeated four times with different aloe vera concentrations as follows:

| Sample Identification | Aloe content |
|---|---|
| B1 | 5 grams |
| B2 | 15 grams |
| B3 | 30 grams |
| B4 | 40 grams |

The time to for each of the above formulations to absorb into the skin was measured as in Example 1 and the time for full absorption is set forth below:

| Sample Identification | Time in seconds |
|---|---|
| B1 | 190 |
| B2 | 45 |
| B3 | 30 |
| B4 | 30 |

### EXAMPLE 10

An emulsion was prepared as follows.

A first solution was formed by dissolving the following ingredients in water and heating the solution with stirring to 65 degrees C. until all ingredients were dissolved:

| | | |
|---|---|---|
| Carbopol Ultrez 10 polymer | 2.4 | grams |
| water | 146.6 | grams |
| Tween 20 surfactant | 5.2 | grams |
| glycerine | 3.0 | grams |
| aqueous aloe solution [1] | 14.0 | grams |

[1] Hawaiian Tropic aloe was used for this experiment.

Four tenths of a gram of allantoin was added to the above solution with stirring and the solution was then permitted to cool to 35 degrees C. Then 3.6 grams of triethanolamine are added to the resulting solution.

A second solution was prepared having the following composition:

| | |
|---|---|
| sodium ascorbyl phosphate | 1.0 grams |
| EDTA, tetrasodium salt | 0.2 grams |

A third solution was prepared having the following composition:

| | |
|---|---|
| flaxseed oil | 1.0 grams |
| Vitamin E | 1.0 grams |
| germaben | 1.0 grams |
| phenagon | 1.0 grams |
| Leciprime S lecithin | 16.8 grams |

Solutions 2 and 3 were mixed with solution 1 with gentle stirring and 6 drops of fragrance were added together with peach colorant to obtain 200 grams of an emulsion. The emulsion was spread onto the back of a hand and fully absorbed within about 30 seconds.

### EXAMPLE 11

This example represents testing of the formulation of Example 6. Samples of the emulsion of Example 7 were given to 30 test subjects with the following instructions: “Select an area of the body for testing. A dry skin area is preferred. The area may be an elbow, the face, a heel, etc. Note the condition of the skin to be treated before starting the evaluation and respond to inquiry 1. Respond to inquiry 2 seven days following start of the evaluation. Respond to the remaining inquiries at the conclusion of the evaluation. Apply the sample to the selected area of the skin once daily. Use the sample sparingly and rub the sample into the skin to form a thin film over the selected area. Be consistent. Always apply the sample in the same amount to the same area. Continue the process for 14 days or until the sample is consumed, whichever occurs first.”

The following are portions of several of the questions asked of the test subjects:

1. Rate the condition of the selected area of the skin for each of the following characteristics prior to application of the sample to the skin where the number 5 signifies severe and the number 1 signifies none at all. (Please circle one number)

| | severe | | | | none at all |
|---|---|---|---|---|---|
| dryness | 5 | 4 | 3 | 2 | 1 |
| roughness | 5 | 4 | 3 | 2 | 1 |
| flakiness | 5 | 4 | 3 | 2 | 1 |
| redness | 5 | 4 | 3 | 2 | 1 |

Results obtained [average of 30 Evaluators]

| | |
|---|---|
| dryness | 3.6 |
| roughness | 3.7 |
| flakiness | 2.5 |
| redness | 2.2 |

2. Rate the condition of the selected area of the skin for each of the following characteristics 7 days following the first application of the sample to the skin where the number 5 signifies severe and the number 1 signifies none at all. (Please circle one number)

| | severe | | | | none at all |
|---|---|---|---|---|---|
| dryness | 5 | 4 | 3 | 2 | 1 |
| roughness | 5 | 4 | 3 | 2 | 1 |
| flakiness | 5 | 4 | 3 | 2 | 1 |
| redness | 5 | 4 | 3 | 2 | 1 |

Results [average of 30 Evaluators]

| | |
|---|---|
| dryness | 2.3 |
| roughness | 2.4 |
| flakiness | 0.7 |
| redness | 1.7 |

3. Rate the condition of the selected area of the skin for each of the following characteristics one day following the last application of the sample to the skin where the number 5 signifies severe and the number 1 signifies none at all. (Please circle one number):

| | severe | | | | none at all |
|---|---|---|---|---|---|
| dryness | 5 | 4 | 3 | 2 | 1 |
| roughness | 5 | 4 | 3 | 2 | 1 |
| flakiness | 5 | 4 | 3 | 2 | 1 |
| redness | 5 | 4 | 3 | 2 | 1 |

Results [average of 30 Evaluators]

| | |
|---|---|
| dryness | 2.0 |
| roughness | 2.2 |
| flakiness | 1.3 |
| redness | 0.7 |

The following is a portion of an additional question asked of the test subjects following the conclusion of the test:

4. Please circle the number that best describes your response to each of the criteria listed below where the number 5 indicates the most favorable response and the number 1 indicates the least favorable response.

| | Most favorable | | | | least favorable |
|---|---|---|---|---|---|
| resulting skin softness | 5 | 4 | 3 | 2 | 1 |
| sample absorption into | 5 | 4 | 3 | 2 | 1 |
| the skin | | | | | |
| non-greasy feel | 5 | 4 | 3 | 2 | 1 |
| ease of application | 5 | 4 | 3 | 2 | 1 |
| color | 5 | 4 | 3 | 2 | 1 |
| smell | 5 | 4 | 3 | 2 | 1 |
| overall sample | 5 | 4 | 3 | 2 | 1 |
| appearance | | | | | |

Results [average of 30 Evaluators]

| | |
|---|---|
| resulting skin softness | 3.6 |
| sample absorption into the skin | 4.1 |
| non-greasy feel | 4.1 |
| ease of application | 4.2 |
| color | 3.1 |
| smell | 2.7 |

Obvious modifications of the invention may be made without departing from the scope of the invention as described herein.

1. A penetrating skin care composition comprising an admixture of (1) oil phase components in a concentration of from 3 to 35 weight percent of the total emulsion comprising, said oil phase components comprising an emulsifier selected from the group of lecithin, phosphatides, saponosides, or mixtures thereof in a concentration of from 1 to 10 percent by weight of the emulsion; one or more oils in a concentration of from 2 to 15 percent of the emulsion; one or more gel formers in a concentration of from 0.1 to 5 percent by weight of the emulsion; and one or more surfactants in a concentration of from 0.1 to 5 percent by weight of the emulsion; and (2) water comprising the balance of the emulsion.

2. The composition of claim 1 where the emulsifier in the oil phase is lecithin.

3. The composition of claim 1 where the oil concentration varies between about 3 and 8 percent by weight of the total emulsion.

4. The composition of claim 3 where the oil is a polar essential oil.

5. The composition of claim 1 where the gel former is a synthetic polymer.

6. The composition of claim 5 where the polymer is a high molecular weight acrylic acid polymer having polyalkenyl polyether crosslinking agents.

7. A penetrating skin care composition comprising an admixture of (1) oil phase components in a concentration of from 3 to 20 weight percent of the total emulsion comprising, said oil phase components comprising an emulsifier selected from the group of lecithin, phosphatides, saponosides and mixtures thereof, in a concentration of from 2 to 10 percent by weight of the emulsion; one or more oils in a concentration of from 1 to 10 percent of the emulsion, one or more gel formers in a concentration of form 0.1 to 5 percent by weight of the emulsion; one or more surfactants in a concentration of from 0.1 to 5 percent by weight of the emulsion; (2) water in a concentration of from 40 to 80 weight percent of the emulsion; and (3) efficacious additives comprising the balance of the emulsion where the efficacious additives differ from the oil phase components, said composition have a pH varying between 6 and 8.

8. The composition of claim 7 where water comprises from 60 to 75 percent by weight.

9. The composition of claim 7 where the emulsifier in the oil phase is lecithin.

10. The composition of claim 7 where the oil concentration varies between about 3 and 7 percent by weight of the total emulsion.

11. The composition of claim 7 where the oil is a polar vegetable oil.

12. The composition of claim 7 where the gel former is a synthetic polymer.

13. The composition of claim 12 where the polymer is a high molecular weight acrylic acid polymer having polyalkenyl polyether crosslinking agents.

14. The composition of claim 12 where the polymer is Carbopol Ultrez 10.

15. The composition of claim 7 where the efficacious additive is selected from the group consisting of anti-wrinkle, anti-skin atrophy, and skin repair actives; skin barrier repair actives; non-steroidal cosmetic soothing actives; artificial tanning actives and accelerators; sunscreen actives; skin lightening actives; sebum stimulators and inhibitors; protease inhibitors; anti itch additives; hair growth inhibitors; 5-alpha reductase inhibitors; desquamating enzyme enhancers; anti-glycation agents; and aloe.

16. The composition of claim 7 where the additive is aloe.

17. A cosmetic emulsion selected from the group of a water in oil emulsion, and a dual emulsion have an aqueous continuous phase, said emulsion comprising tightly packed micelles having walls in contact with each other where a substantial portion of the micelles are micro spheres of water and dissolved efficacious additives coated with a lecithin-oil containing membrane.

18. The emulsion of claim 17 where the emulsion is rigidified.

19. The emulsion of claim 17 containing aloe.

20. A method for treating the skin, said method comprising coating the skin with a film of the composition of claim 7.
